(19) United States
(12) Patent Application Publication
(10) Pub. No.: US 2015/0023897 A1
(43) Pub. Date: Jan. 22, 2015

(54) METHOD OF PROTECTING SKIN FROM OXIDATIVE STRESS

(71) Applicant: The Procter & Gamble Company, Cincinnati, OH (US)

(72) Inventor: John Erich OBLONG, Loveland, OH (US)

(21) Appl. No.: 14/309,074

(22) Filed: Jun. 19, 2014

Related U.S. Application Data
(60) Provisional application No. 61/856,972, filed on Jul. 22, 2013.

(51) Int. Cl. A61K 8/368; A61K 8/49; A61Q 19/08; A61Q 17/04

(52) U.S. Cl. CPC A61K 8/368; A61Q 17/04; A61K 8/4926; A61Q 19/08; A61K 2800/592
USPC 424/60

(57) ABSTRACT

A method of protecting skin from an environmental stressor that includes identifying a target skin portion where protection from environmental stress is desired and topically applying a suitable skin care composition thereto. The skin composition may include a safe and effective amount of a vitamin B₃ compound, a safe and effective amount of a salicylate or a suitable derivative of salicylate, an organic sunscreen active, and a dermatologically acceptable carrier. The composition may have a pH of between about 5 and about 7, and is left on the target skin portion for a sufficient amount of time for the vitamin B₃ compound and salicylate to provide protection from the environmental stressor.

### FIELD

The present disclosure is directed, generally, to a method of protecting skin cells from oxidative stress and compositions therefor. More specifically, a method of protecting skin from the effects of oxidative stress on skin cells by topically applying a composition comprising effective amounts of niacinamide and salicylate to the skin is disclosed.

### BACKGROUND

As people age, intrinsic factors related to the biochemical changes within the skin typically result in visible signs of skin aging such as wrinkling and other forms of roughness (including increased pore size, flaking and skin lines), uneven skin pigmentation (e.g., age spots or melasma) as well as other histological changes associated with skin aging. In addition, lifestyle choices and exposure to the environment may allow extrinsic factors such as ultraviolet radiation, pollution (e.g., engine exhaust, cigarette smoke, smog), wind, heat, low humidity, harsh surfactants, abrasives, and the like to damage the skin, which may lead to premature skin aging and the tell-tale visible signs that accompany it.

To many people, the visible signs of skin aging are an undesirable reminder of the disappearance of youth. As a result, the elimination of wrinkles has become a booming business in youth-conscious societies. Treatments range from cosmetic creams and moisturizers to various forms of cosmetic surgery. Extrinsic or intrinsic factors may result in the thinning and general degradation of the skin. For example, as the skin naturally ages, it is not uncommon to observe a reduction in the cells and blood vessels that supply the skin or a flattening of the dermal-epidermal junction.

A multitude of skin care products have been developed to improve the visible signs of aging skin. And it is not uncommon for conventional skin care products to include one or more sunscreen agents to protect the skin from the harmful effects of UV radiation. However, there remains a need to provide a personal care composition that protects the skin from the harmful effects of other environmental stressors as well as UV radiation. There is also a need to provide a personal care composition that protects the skin from the undesirable effects of environmental stressors such as UV radiation even after the composition is removed.

### BRIEF DESCRIPTION OF THE DRAWINGS

FIG. 1 is an illustration of UV Redness Scores observed in Clinical 1.

FIG. 2 is an illustration of overall average UV Redness Scores observed in Clinical 1.

FIG. 3 is an illustration of the UV Redness Scores observed in Clinical 1 one day after UV exposure.

FIG. 4 is an illustration of the a* Values observed in Clinical 1.

FIG. 5 is an illustration of the overall a* Values observed in Clinical 1.

FIG. 6 is an illustration of the a* Values observed in Clinical 1 one day after exposure to UV.

FIG. 7 is an illustration of the overall L* values observed in Clinical 1.

FIG. 8 is an illustration of the change in a* Values from basal observed in Clinical 2.

FIG. 9 is an illustration of the change in a* Values observed in Clinical 2 two days after exposure to UV.

FIG. 10 is an illustration of the overall average Redness Score observed in Clinical 2.

### SUMMARY

In order to provide a solution to the problems above, disclosed herein is a method of protecting skin from an environmental stressor. In some instances, the method comprises identifying a target skin portion where protection from environmental stress is desired and topically applying a skin care composition thereto. In some instances, The skin composition includes a safe and effective amount of a vitamin B₃ compound, a safe and effective amount of a salicylate, an organic sunscreen active, and a dermatologically acceptable carrier. The composition has a pH of between about 5 and about 7 and is left on the target skin portion for a sufficient amount of time for the niacinamide and salicylate to provide protection from the environmental stressor.

In another embodiment, a method of protecting skin from UV induced erythema is disclosed. The method comprising: identifying a target skin portion where protection from UV induced erythema is desired and topically applying a skin care composition to the target skin area. The skin care composition comprises a safe and effective amount of niacinamide, a safe and effective amount of a salicylate, an organic sunscreen active, a dermatologically acceptable carrier, and a pH of between about 5 and about 7; and allowing the skin care composition to remain on the target skin portion for a sufficient amount of time for the niacinamide and salicylate to provide protection from erythema.

In another embodiment, a method of protecting skin from UV induced erythema is disclosed. The method comprising: identifying a target skin portion where protection from UV induced erythema is desired and topically applying a skin care composition to the target skin area. The skin care composition comprises a safe and effective amount of niacinamide, a safe and effective amount of a salicylate derivative selected from the group consisting of 3,4-dihydroxybenzoate, 2,5-dihydroxybenzoate, 2,6-dihydroxybenzoate, and salts of rhein, an organic sunscreen active, a dermatologically acceptable carrier, and a pH of between about 5 and about 7; and allowing the skin care composition to remain on the target skin portion for a sufficient amount of time for the niacinamide and salicylate to provide protection from erythema.

### DETAILED DESCRIPTION

All percentages are by weight of the personal-care composition, unless otherwise specified. All ratios are weight ratios, unless specifically stated otherwise. All numeric ranges are inclusive of narrower ranges; delineated upper and lower range limits are interchangeable to create further ranges not explicitly delineated. The number of significant digits conveys neither limitation on the indicated amounts nor on the accuracy of the measurements. All measurements are understood to be made at about 25° C. and at ambient conditions, where “ambient conditions” means conditions under about one atmosphere of pressure and at about 50% relative humidity. Also, as used in the specification, including the appended claims, the singular forms “a,” “an,” and “the” include the plural, and reference to a particular numerical value includes at least that particular value, unless the context clearly dictates otherwise.

### Definitions.

“Cosmetic” means providing a desired visual effect on an area of the human body. The visual cosmetic effect may be temporary, semi-permanent, or permanent. Some non-limiting examples of “cosmetic products” include products that leave color on the face, such as foundation, mascara, concealers, eye liners, brow colors, eye shadows, blushers, lip sticks, lip balms, face powders, solid emulsion compact, and the like.

“Dermatologically acceptable” means that the compositions or components thereof so described are suitable for use in contact with human skin without undue toxicity, incompatibility, instability, allergic response, and the like.

“Disposed” means an element is positioned in a particular place relative to another element.

“Environmental Stressor” means an environmental element that causes the formation of undesirable reactive oxygen species in a cell, which cause oxidative damage to cellular components. Additionally, environmental stressor can elicit an inflammatory response. Non-limiting examples of environmental stressors include ultraviolet radiation (UV-A and UV-B), cigarette and cigar smoke, ozone, combustible engine exhaust, diesel exhaust, smog, surfactants, and radiation from a computer, telephone, or television monitor.

“Erythema” means redness of the skin caused by an environmental stressor such as UV radiation (e.g., “sunburn”).

“Protect” and variations thereof, when referring to the environmental stress benefit(s) provided by the compositions herein, mean to reduce or eliminate the effect of an environmental stressor on skin. For example, when referring to a UV protection benefit, the composition or active component(s) thereof may reduce or eliminate damage and/or the appearance of damage to skin caused by UV-A and/or UV-B radiation. Non-limiting examples of such protection include reducing or eliminating erythema, reducing edema or localized swelling, reduced inflammatory response, blocking glycation product formulation, and mitigating bather disruption.

“Regulate a skin condition” means maintaining skin appearance and/or feel with little to no degradation in appearance and/or feel.

“Safe and effective amount” means an amount of a compound or composition sufficient to significantly induce a positive benefit such as a positive skin or feel benefit, including independently or in combinations the benefits disclosed herein, but low enough to avoid serious side effects, i.e., to provide a reasonable benefit to risk ratio, within the scope of sound judgment of the skilled artisan. For example, in some instances, a safe and effective amount of niacinamide and sodium salicylate (alone or in combination with one another) may mean an amount suitable for reducing or eliminating erythema in skin exposed to UV radiation within 24 hours after application of a composition comprising the niacinamide and salicylate.

“Skin” means the outermost protective covering of mammals that is composed of cells such as keratinocytes, fibroblasts and melanocytes. Skin includes an outer epidermal layer and an underlying dermal layer. Skin may also include hair and nails as well as other types of cells commonly associated with skin, such as, for example, myocytes, Merkel cells, Langerhans cells, macrophages, stem cells, sebocytes, nerve cells and adipocytes.

“Skin-care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin bather properties, improve skin tone, reducing the appearance of redness or skin blotches, and/or improving the brightness, radiancy, or translucency of skin. Some nonlimiting examples of “skin-care products” include skin creams, moisturizers, lotions, and body washes.

“Skin-care composition” means a composition that regulates and/or improves skin condition.

“Skin-care active” means a compound or combination of compounds that, when applied to skin, provide an acute and/or chronic skin care benefit or to a type of cell commonly found in skin. Skin-care actives may regulate and/or improve skin or its associated cells (e.g., improve skin elasticity; texture, tone, hydration, overall appearance and cell metabolism).

“Topical application” means to apply or spread the compositions of the present invention onto the surface of the keratinous tissue.

“Treated,” as used herein, refers to a target portion of skin to which one or more of the compositions disclosed herein have been applied within the past 24 hours.

“Untreated,” as used herein, refers to a target portion of skin to which one or more of the compositions disclosed herein have not been applied.

As humans age, damage from external and internal stressors on the cells of the body accumulates (i.e., oxidative stress), which may lead to decreased efficiency and function of tissue and organs. In skin, this damage may manifest as visible signs of aging. Surprisingly, it has been found that skin care compositions containing effective amounts of a vitamin B₃ compound and a salicylate provide improved protection from at least some damaging environmental stressors when compared to compositions that only contain one of these compounds.

Composition

The compositions herein include a safe and effective amount of a vitamin B₃ compound, a safe and effective amount of a salicylate and a sunscreen agent. Vitamin B₃ compounds and salicylic acid are known for use in conventional cosmetic compositions to combat the visible signs of aging in skin. However, conventional cosmetic compositions typically include vitamin B₃ compounds and/or salicylic acid to reverse the visible signs of aging in skin caused by past intrinsic and/or extrinsic insults. Until now, it was unknown that including an effective amount of a salicylate and an effective amount of a vitamin B₃ compound in a topical cosmetic composition may protect skin from future cellular damage associated with environmental stressors. For example, it has been surprisingly discovered that a vitamin B₃ compound and salicylate, when topically applied to skin, may work together to protect skin from erythema caused by exposure to UV radiation. In particular, it has been discovered that compositions that include an effective amount of a vitamin B₃ compound and salicylate continue to combat the undesirable effects of environment stress even after the composition has been removed. It is believed, without being limited by theory, that the vitamin B₃ compound and salicylate may affect different biochemical pathways, thereby enabling them to work together to provide an improved environmental protection benefit.

As discussed in more detail below, the environmental protection benefit provided by an effective amount of a vitamin B₃ compound and salicylate may enable formulations that require less sunscreen active and/or less potent sunscreen active. In addition, a user of the present composition and method who skips a step in their daily skin care regiment (i.e., does not apply the present composition) or forgoes their regimen altogether may still be protected from some of the effects of environmental stress. Some examples of compositions suitable for use herein and methods of making the same are described in co-pending U.S. Provisional Ser. No. 61/856,861, filed by Gantley, et al., on Jul. 22, 2013 and titled “Stable High Salt Containing Skin Care Compositions.”

Salicylate and Vitamin B₃ Compound

Salicylates are commonly recognized as being the salts and esters of salicylic acid (e.g., sodium salicylate). Salicylic acid is well known for its use as an anti-inflammatory and to exfoliate skin, and it is not uncommon to find salicylic acid in a conventional cosmetic composition. In particular, it may be added to skin care compositions to help exfoliate the outer layers of the stratum corneum, which can improve the appearance of the skin. However, in order to provide an exfoliation and/or anti-inflammatory benefit, it is commonly believed that the salicylic acid must be in its acid form when applied to the skin, as opposed to the salicylate form of the present composition. But because human skin typically has a pH of around 5.5 (e.g., 5.4-5.9), salicylic acid is converted to its salicylate form relatively quickly. Thus, in order for conventional skin care compositions to introduce a sufficient amount of salicylic acid to the skin to provide the desired benefit, they are typically formulated at relatively low pH (e.g., between 2.5 and 5.0 or about 3.5). Formulating at such a relatively low pH enables the inclusion of a suitable amount of salicylic acid, but it can create other problems.

One problem associated with formulating at low pH is reducing the activity of pH sensitive skin care actives in the composition or even rendering these compounds ineffective for providing a desired skin care benefit. For example, certain vitamin B₃ compounds such as niacinamide are pH sensitive and tend to form complexes in acid environments. In some instances, these complexes may have a significantly reduced ability to deliver the desired skin care benefit or may not exhibit any beneficial activity at all.

Another problem associated with formulating at low pH is that the composition may cause skin irritation to some users and/or undesirably affect the skin commensal microorganisms necessary for maintaining healthy skin.

Thus, utilizing salicylate as opposed to salicylic acid allows the present composition to be formulated at relatively higher pH (e.g., greater than 5) and avoids some of the problems of low pH compositions. Of course, it is to be appreciated that topical skin care compositions having a pH of greater than 7 may also cause undesirable skin irritation to some users or have other undesirable drawbacks. Thus, it is important to ensure that the pH of the present composition is higher than 5, but typically less than or equal to about 7 (e.g., from 5.0 to 7.5 or even from 5.1 to 6.0).

In addition, the discovery of the environmental protection benefits of salicylate disclosed herein contrast with the conventional thinking that the salicylic acid form of this material is necessary to provide a skin health benefit. Thus, it is believed, without being limited by theory, that salicylate may act via a different biological mechanism.

In certain embodiments, a pre-made salicylate may be added directly to the present composition or one or more phases thereof (e.g., in the case of a multiphase composition such as an emulsion) during formulation of the composition. Additionally or alternatively, salicylic acid may be added to the composition and converted to a salicylate in situ (e.g., by adding a sufficient amount of NaOH or other suitable reactant, which is within the level of skill of one skilled in the art). The present compositions may include salicylate at 0.001% to 10% by weight based on the weight of the composition (e.g., from 0.1 wt % to 5 wt %, from 0.25 wt % to 3 wt %, or from 0.5 wt % to 1.5 wt %). In some instances, salicylate derivatives such as 3,4-dihydroxybenzoate, 2,5-dihydroxybenzoate, 2,6-dihydroxybenzoate, and salts of rhein (sometimes referred to as cassic acid or 4,5-dihydroxy-9,10-dioxoanthracene-2-carboxylic acid) may be suitable for use herein.

The compositions herein include a safe and effective amount of a vitamin B₃ compound. In some instances, the composition may include from 0.01% to 50% by weight of the vitamin B₃ compound (e.g., from 0.1% to 10%, from 0.5% to 10%, from 1% to about 5%, or even from 2% to 5%). “Vitamin B₃ compound” means a compound having the formula:

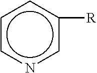

wherein R is —CONH₂ (i.e., niacinamide), —COOH (i.e., nicotinic acid) or —CH₂OH (i.e., nicotinyl alcohol); derivatives thereof; and salts of any of the foregoing. Some non-limiting examples of derivatives include nicotinic acid esters, nicotinyl amino acids, nicotinyl alcohol esters of carboxylic acids, nicotinic acid N-oxide and niacinamide N-oxide. Esters of nicotinic acid include nicotinic acid esters of C₆-C₂₂, (e.g., C₁-C₁₆ alcohols or C₁-C₆ alcohols). The alcohols are suitably straight-chain or branched chain, cyclic or acyclic, saturated or unsaturated (including aromatic), and substituted or unsubstituted. In certain embodiments, the esters are “non-vasodilating,” which means that the ester does not commonly yield a visible flushing response after application to the skin in the subject compositions (i.e., the majority of the general population would not experience a visible flushing response, although such compounds may cause vasodilation not visible to the naked eye). In certain embodiments, a nicotinic acid material that is rubifacient at higher doses could be used at a lower dose to reduce or eliminate the rubifacient effect. Some non-vasodilating esters of nicotinic acid include tocopherol nicotinate and inositol hexanicotinate. Some other derivatives of the vitamin B₃ compound include derivatives of niacinamide resulting from substitution of one or more of the amide group hydrogens and include nicotinyl amino acids, derived, for example, from the reaction of an activated nicotinic acid compound (e.g., nicotinic acid azide or nicotinyl chloride) with an amino acid and nicotinyl alcohol esters of organic carboxylic acids (e.g., C₁-C₁₈). Some specific examples of such derivatives include nicotinuric acid (C₈H₈N₂O₃) and nicotinyl hydroxamic acid (C₆H₆N₂O₃), which have the following chemical structures:

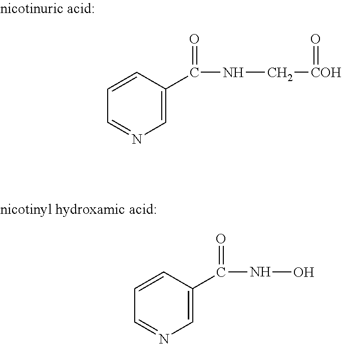

Some exemplary nicotinyl alcohol esters include nicotinyl alcohol esters of the carboxylic acids salicylic acid, acetic acid, glycolic acid, palmitic acid and the like. Other non-limiting examples of vitamin B₃ compounds useful herein are 2-chloronicotinamide, 6-aminonicotinamide, 6-methylnicotinamide, n-methyl-nicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl)urea, 2-mercaptonicotinic acid, nicomol, and niaprazine.

Vitamin B₃ compounds are commercially available from a number of sources, e.g., the Sigma Chemical Company (St. Louis, Mo.); ICN Biomedicals, Inc. (Irvin, Calif.) and Aldrich Chemical Company (Milwaukee, Wis.).

In some instances, it may be desirable for the ring nitrogen of the vitamin B₃ compound to be “uncomplexed” (i.e., chemically free (e.g., unbound and/or unhindered)) in the composition and/or prior to application to a target skin surface. For example, the compositions herein may be “relatively free” of a salt or complex of a vitamin B₃ compound such as niacinamide (i.e., the composition contains less than 50%, 40%, 30%, 20%, 10% or even less than 5% of a salt or complex of vitamin B₃). The composition may be “substantially free” of a salt or complex of a vitamin B₃ compound (i.e., less than 3%, 2%, 1% or even less than 0.5%) or “completely free” of a salt or complex of a vitamin B₃ compound (i.e., 0%). Exemplary approaches to minimizing or preventing the formation of undesirable salts and/or complexes include omission of materials that form substantially irreversible or other undesirable complexes with the vitamin B₃ compound in the composition, pH adjustment, ionic strength adjustment, the use of surfactants, and practicing formulation processes wherein the vitamin B₃ compound and materials which complex therewith are in different phases. Such approaches would be recognized by one skilled in the art.

In some instances, organic and/or inorganic salts of the vitamin B₃ compound may be useful herein. Some nonlimiting examples of inorganic salts of the vitamin B₃ compound include inorganic salts with anionic inorganic species (e.g., chloride, bromide, iodide, carbonate, preferably chloride). Some nonlimiting examples of organic salts of the vitamin B₃ compound include organic carboxylic acid salts such as mono-, di- and tri-C₁-C₁₈ carboxylic acid salts (e.g., acetate, salicylate, glycolate, lactate, malate, citrate, and monocarboxylic acid salts such as acetate). These and other salts of the vitamin B₃ compound can be readily prepared by the skilled artisan.

In some instances, it may be desirable for the vitamin B₃ compound to be relatively, substantially or completely uncomplexed prior to and/or upon delivery to the skin. Thus, in embodiments wherein the vitamin B₃ compound is in the form of a salt or otherwise complexed form (before and/or after addition to the composition), such complex is substantially reversible at a pH of from about 5.0 to about 7.0 (e.g., upon delivery of the composition to skin). Such reversibility can be readily determined by one skilled in the art.

Sunscreen Active

The present composition includes one or more sunscreen actives. Herein, “sunscreen active” includes both sunscreen agents and physical sunblocks. Sunscreen actives may be organic or inorganic. However, inorganic sunscreen actives such as titanium dioxide and zinc oxide are known to form a visible white film on skin, especially when included at levels sufficient to provide Sun Protection Factor (“SPF”) suitable for the present composition (e.g., SPF 5 or greater, SPF 10 or greater, SPF 15 or greater, SPF 20 or greater or even SPF 30 or greater, but typically less than SPF 50). Many consumers find the visible white film formed by inorganic sunscreens to be undesirable, especially for skin care products that are used as part of a daily beauty regimen. Accordingly, in some instances, it may be desirable to include only organic sunscreen actives in the compositions herein. For example, the present compositions may include from 1% to 20% or even from 2% to 10% by weight, of an organic sunscreen active. Exact amounts will vary depending upon the chosen sunscreen active and the desired Sun Protection Factor (SPF), and are within the knowledge and judgment of one skilled in the art.

Examples of sunscreen actives and ultraviolet light absorbers are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93. Particularly suitable organic sunscreen actives include 2-ethylhexyl-p-methoxycinnamate (commercially available as PARSOL™ MCX), 4,4′-t-butyl methoxydibenzoyl-methane (commercially available as PARSOL™ 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, menthyl anthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, benzylidene camphor and derivatives thereof.

Dermatologically Acceptable Carrier

The compositions herein may include a dermatologically acceptable carrier (“carrier”) that provides a suitable matrix to store and deliver the actives (i.e., salicylate, vitamin B₃ compound and sunscreen) and other optional ingredients. The phrase “dermatologically acceptable carrier”, as used herein, means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. Non-limiting examples include simple solutions (e.g., aqueous, organic solvent, or oil based), emulsions, and solid forms (e.g., gels, sticks, flowable solids, or amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil and oil-in-water-in-oil). The oil phase herein may comprise silicone oils, non-silicone oils such as hydrocarbon oils, esters, ethers, and the like, and mixtures thereof.

The aqueous phase typically comprises water. However, in other embodiments, the aqueous phase may comprise components other than water, including but not limited to water-soluble moisturizing agents, conditioning agents, anti-microbials, humectants and/or other water-soluble skin care actives. In one embodiment, the non-water component of the composition comprises a humectant such as glycerin and/or other polyols. However, it should be recognized that the composition may be substantially (i.e., less than 1% water) or fully anhydrous.

A suitable carrier is selected to yield a desired product form. Furthermore, the solubility or dispersibility of the components (e.g., extracts, sunscreen active, additional components) may dictate the form and character of the carrier. In one embodiment, an oil-in-water or water-in-oil emulsion is preferred.

Emulsions may further comprise an emulsifier. The composition may comprise any suitable percentage of emulsifier to sufficiently emulsify the carrier. Suitable weight ranges include from about 0.1% to about 10% or about 0.2% to about 5% of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986). Suitable emulsions may have a wide range of viscosities, depending on the desired product form.

The carrier may further comprise a thickening agent as are well known in the art to provide compositions having a suitable viscosity and rheological character.

### Optional Components

In some embodiments, the inventive composition herein may include one or more optional components known for use in topical cosmetic compositions, provided the optional components do not unacceptably alter the desired benefits of the composition. The optional components, when present, may be included at an amount of about 50%, 40%, 30%, 20%, 10%, 5%, or 3%, by weight of the composition, for example, at least about 0.001%, 0.01%, 0.1%, 0.2%, 0.5%, or 1%, by weight of the composition. Suitable ranges include any combination of the lower and upper limits including suitable ranges from about 0.1% to about 50%; from about 0.2% to about 20%; or from about 1% to about 10%, by weight of the composition.

The optional components, when incorporated into the composition, should be suitable for use in contact with human skin tissue without undue toxicity, incompatibility, instability, allergic response, and the like. Nonlimiting examples of optional components include skin anti-aging agents, skin tone agents, anti-inflammatory agents, anti-acne actives, desquamation actives, anti-cellulite agents, chelating agents, flavonoids, tanning active, non-vitamin antioxidants and radical scavengers, hair growth regulators, anti-wrinkle actives, anti-atrophy actives, minerals, phytosterols and/or plant hormones, N-acyl amino acid compounds, antimicrobial or antifungal actives, and other useful skin care actives, which are described in further detail in U.S. Publication Nos. US2006/0275237A1 and US2004/0175347A1.

### II. Methods of Use

Various methods of treatment, application, regulation, or improvement may utilize the aforementioned compositions. Identification of a region of skin in need of protection from environmental stressors (e.g., skin that includes visible signs of aging) may occur on any skin surface of the body. Skin surfaces of the most concern tend to be those not typically covered by clothing such as facial skin surfaces, hand and arm skin surfaces, foot and leg skin surfaces, and neck and chest skin surfaces. In particular, identification of a region of skin in need of protection may be on a facial skin surface such as the forehead, perioral, chin, periorbital, nose, and/or cheek skin surfaces. The target skin surface may include any visible sign of skin aging known in the art (e.g., fine lines, deep lines, wrinkles, course texture, sagging, unfirm skin and lack of elasticity), but does not necessarily need to contain such features. For example, a relative young user who has not yet developed visible signs of aging may still wish to target areas of skin that typically develop signs of aging later in life. The composition may be applied to the target skin portion and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed.

The treatment period is ideally of sufficient time for the vitamin B₃ compound and salicylate to provide a skin protection benefit (e.g., skin appearance, texture and/or cell metabolism benefit after exposure to an environmental stressor such as UV). The treatment period may be at least 1 week, and in certain embodiments the treatment period may last about 2weeks, 4 weeks, 8 weeks, or 12 weeks. In certain embodiments, the treatment period will extend over multiple months (i.e., 3-12 months) or multiple years. In certain embodiments the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., wrinkles around the eyes) while minimizing delivery to skin surface not requiring treatment. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

In some instances, the method herein comprises identify a target area of skin that is generally exposed to UV (i.e., not covered by clothing) for a substantial portion of the day (e.g., more than 4, 6, 8, 10, or even 12 hours), and applying a composition comprising an effective amount of a vitamin B₃ compound, an effective amount of salicylate and an organic sunscreen agent to the target area of skin. In these embodiments, the effective amount of vitamin B₃ compound and salicylate is demonstrated as being present when the identified area of skin is exposed to an amount of UV radiation sufficient to induce erythema and the erythema is less than what would be observed by a composition that does not include the vitamin B₃ compound and salicylate.

### EXAMPLES

### Example 1

### Exemplary Compositions

Tables 1 and 2 set forth some non-limiting examples of compositions suitable for use herein. Salicylic acid is shown as being included in the exemplary compositions, but it is to be appreciated that the pH of the final composition is adjusted to be 6.0 or about 6.0 so that salicylate will be present in the composition. The examples provided in Table 1 are solely for the purpose of illustration and are not to be construed as limitations herein, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minor materials will vary depending on the physical and chemical characteristics of the particular ingredients selected to make the present invention as described herein.

The exemplary compositions may be generally prepared according to conventional methods known in the art of making compositions and topical compositions. Such methods typically involve mixing of ingredients in one or more steps to a relatively uniform state, with or without heating, cooling, application of vacuum, and the like. For example, emulsions may be prepared by first mixing the aqueous phase materials separately from the fatty phase materials and then combining the two phases as appropriate to yield the desired continuous phase. In some instances, the compositions may be prepared to provide suitable stability (physical stability, chemical stability, photostability, etc.) and/or delivery of active materials. The composition may be provided in a package sized to store a sufficient amount of the composition for a treatment period.

TABLE 1
Exemplary Compositions

| | Wt % | | | | | |
|---|---|---|---|---|---|---|
| Component | A | B | C | D | E | F |
| Water Distilled | 76.060 | 73.950 | 73.450 | 75.56 | 72.950 | 75.06 |
| Glycerin | 5.000 | 5.000 | 5.000 | 5.000 | 5.000 | 5.000 |
| Salicylic Acid | 0.500 | 1.500 | 1.500 | 0.500 | 1.500 | 0.500 |
| Triethanolamine | 0.540 | 1.650 | 1.650 | 0.540 | 1.650 | 0.540 |
| EDTA (Na₂—S) | 0.100 | 0.100 | 0.100 | 0.100 | 0.100 | 0.100 |
| Tocopheryl Acetate | 0.500 | 0.500 | 0.500 | 0.500 | 0.500 | 0.500 |
| Idebenone | — | — | 0.500 | 0.500 | 1.000 | 1.000 |
| Isohexadecane | 3.000 | 3.000 | 3.000 | 3.000 | 3.000 | 3.000 |
| Isopropyl Isostearate | 1.330 | 1.330 | 1.330 | 1.330 | 1.330 | 1.330 |
| SEFA Cottonate | 0.670 | 0.670 | 0.670 | 0.670 | 0.670 | 0.670 |
| TOSPEARL 2000 B | 0.250 | 0.250 | 0.250 | 0.250 | 0.250 | 0.250 |
| EMULGADE PL 68/50 | 0.200 | 0.200 | 0.200 | 0.200 | 0.200 | 0.200 |
| Behenyl Alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl Alcohol | 0.320 | 0.320 | 0.320 | 0.320 | 0.320 | 0.320 |
| Stearyl Alcohol | 0.480 | 0.480 | 0.480 | 0.480 | 0.480 | 0.480 |
| PEG-100 stearate | 0.100 | 0.100 | 0.100 | 0.100 | 0.100 | 0.100 |
| Sepigel 305 | 2.000 | 2.000 | 2.000 | 2.000 | 2.000 | 2.000 |
| D-Panthenol | 1.000 | 1.000 | 1.000 | 1.000 | 1.000 | 1.000 |
| Niacinamide USP-FCC | 5.000 | 5.000 | 5.000 | 5.000 | 5.000 | 5.000 |
| Benzyl alcohol | 0.250 | 0.250 | 0.250 | 0.250 | 0.250 | 0.250 |
| Dow Corning 1503 Fluid | 2.000 | 2.000 | 2.000 | 2.000 | 2.000 | 2.000 |
| pH | ~6.0 | ~6 | ~6 | ~6.0 | ~6 | ~6.0 |

TABLE 2
Exemplary Compositions

| | Wt % | | | | | |
|---|---|---|---|---|---|---|
| Component | G | H | I | J | K | L |
| water | qs | qs | qs | qs | qs | qs |
| glycerol | 5.0000 | 7.0000 | 7.0000 | 10.0000 | 5.0000 | 10.0000 |
| phenylbenzimidazole | 1.2500 | 1.2500 | 1.2500 | 1.2500 | 1.2500 | 1.2500 |
| sulfonic acid | | | | | | |
| disodium EDTA | 0.1000 | 0.1000 | 0.1000 | 0.1000 | 0.1000 | 0.1000 |
| Idebenone | — | — | 0.500 | 0.500 | 1.000 | 1.000 |
| Salicylic Acid | 0.500 | 1.500 | 1.500 | 0.500 | 1.500 | 0.500 |
| triethanolamine | 0.7500 | 0.7500 | 0.7500 | 0.7500 | 0.7500 | 0.7500 |
| sodium metabisulfite | 0.1000 | 0.2000 | 0.1000 | 0.1000 | 0.1000 | 0.1000 |
| BHT | 0.0150 | 0.0150 | 0.0150 | 0.0150 | 0.0150 | 0.0150 |
| titanium dioxide | 0.2500 | 0.4500 | 0.4500 | 0.7500 | 0.5500 | 0.4500 |
| niacinamide | 2.0000 | 2.0000 | 2.0000 | 3.5000 | 5.0000 | 3.5000 |
| dexpanthenol | 0.25 | 0.5000 | 1.0000 | 2.0000 | 1.0000 | 1.0000 |
| C12-C15 alkyl benzoate | 5.00 | 2.5000 | 1.5000 | 2.5000 | 0 | 2.5000 |
| caprylic/capric triglyceride | 1.0 | 1.5000 | 1.5000 | 1.5000 | 1.5000 | 1.5000 |
| octyl salicylate | 5.0000 | 5.0000 | 5.0000 | 5.0000 | 5.0000 | 5.0000 |
| octocrylene | 1.0000 | 1.0000 | 1.0000 | 1.0000 | 1.0000 | 1.0000 |
| butyl | 2.0000 | 2.0000 | 2.0000 | 2.0000 | 2.0000 | 2.0000 |
| methoxydibenzoylmethane | | | | | | |
| cetyl alcohol | 0.5000 | 0.5000 | 0.5000 | 0.5000 | 0.5000 | 0.5000 |
| tocopherol acetate | 0.5000 | 0.5000 | 0.5000 | 0.5000 | 0.5000 | 0.5000 |
| sorbitan stearate/sucrose | 1.0000 | 1.0000 | 1.0000 | 1.0000 | 1.0000 | 1.0000 |
| cocoate | | | | | | |
| cetearyl glucoside | 0.5000 | 0.5000 | 0.5000 | 0.5000 | 0.5000 | 0.5000 |
| stearyl alcohol | 0.7000 | 0.7000 | 0.7000 | 0.7000 | 0.7000 | 0.7000 |
| behenyl alcohol | 0.6000 | 0.6000 | 0.6000 | 0.6000 | 0.6000 | 0.6000 |
| ethyl paraben | 0.2000 | 0.2000 | 0.2000 | 0.2000 | 0.2000 | 0.2000 |
| propyl paraben | 0.1000 | 0.1000 | 0.1000 | 0.1000 | 0.1000 | 0.1000 |
| PEG-100 stearate | 0.1000 | 0.1000 | 0.1000 | 0.1000 | 0.1000 | 0.1000 |
| polymethylsilsesquioxane | 0.2500 | 0.5000 | 1.5000 | 0.5000 | 0.2500 | 0.5000 |
| Phase C | | | | | | |
| polyacrylamide/C13-14 | 2.000 | 2.2500 | 2.5000 | 2.5000 | 3.0000 | 2.5000 |
| isoparaffin/laureth-7 | | | | | | |
| benzyl alcohol | 0.2500 | 0.2500 | 0.2500 | 0.2500 | 0.2500 | 0.2500 |
| perfume | 0.2000 | 0.2000 | 0.2000 | 0.2000 | 0.2000 | 0.2000 |

### Example 2

### Clinical Study I

This example illustrates an environmental protection benefit provided by the present composition on the skin of female test subjects. This objective of the study is to assess the ability of a composition comprising niacinamide and salicylate to prevent and/or reduce UV-induced changes in the skin. The assessment of performance level benefits was evaluated across metrics of pigmentation (erythema, L*a*b*). The primary endpoint is redness (via visual grading and measured a* Values and L* Values).

Fifty-five female test subjects aged 20-45 years were recruited. The test subjects were identified as having Fitzpatrick Skin Type II or III. The study has 80% power to detect a treatment difference of 0.13 on the UV Redness measurement using a two-sided test at an alpha-level of 0.05. The study is a four week study employing a complete block design in which the different product treatments (or no treatment) are evaluated on twelve 3×3 cm sites on a subject's back. There is a brief wash out period (2 days) followed by approximately 2 weeks of daily application of the test compositions done at the study facility. On day 16, the test sites on each subject's back are exposed to a single controlled UV dose of 1.5 times the Minimal Erythemal Dose (“MED”) prior to applying any of test compositions to their respective treatment sites. MED is a well known metric used to define the threshold UV exposure dose that will produce erythema. After the MED of UV, the test compositions were applied to their respective test sites. The test compositions are applied daily for 9 days after the UV exposure (i.e., days 17-26). On days 27-30, no compositions were applied to any of the test sites, and measurements were obtained to observe the resolution of the UV induced erythema. Subjects were required to use only the provided cleansing product (OLAY brand bar soap) for bathing throughout the study. Test products were applied to each treatment site at 3 mg/cm² by facility trained staff. There were no treatment applications on weekends. The results of the test are discussed below with reference to the figures.

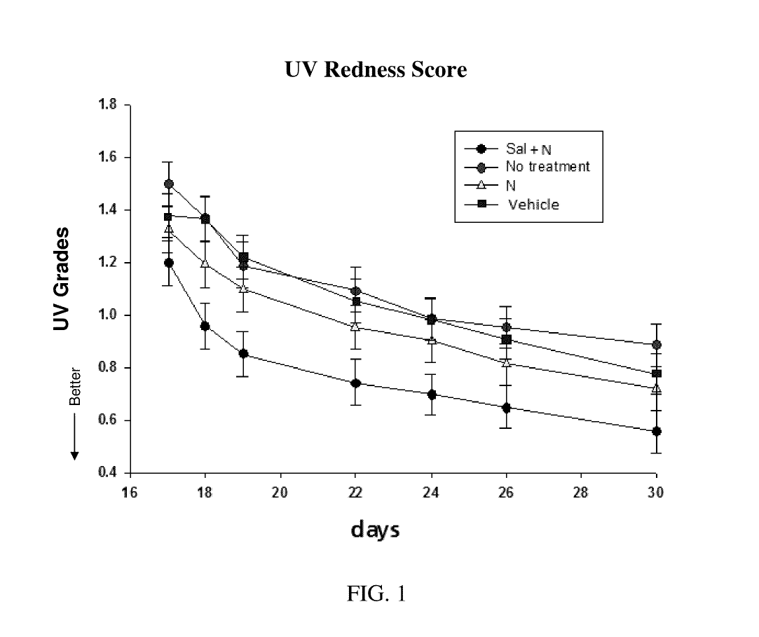

FIG. 1 shows a plot of the UV redness score from day 17 to day 30 for each of four treatment sites. Three of the four treatment sites were each treated with one of the following compositions: (i) vehicle, (ii) vehicle+5% niacinamide, or (iii) vehicle+5% niacinamide+1.5% salicylate (Composition B in Table 1). The fourth treatment site was not treated with a composition. The redness score of each test site was determined visually by an expert grader prior to applying the composition. As illustrated in FIG. 1, the composition containing the niacinamide and salicylate provided the lowest UV redness score, which is believed to correspond to lower UV induced erythema. It is especially important to note that prior to the application of any of the compositions on day 17, the niacinamide/salicylate treatment site provided the best protection from UV induced erythema as demonstrated by a lower initial UV redness score. In addition, at the end of the 30 day observation period, the niacinamide/salicylate containing composition had the lowest UV redness score, indicating that it may also aid in the recovery of the skin from the UV induced erythema.

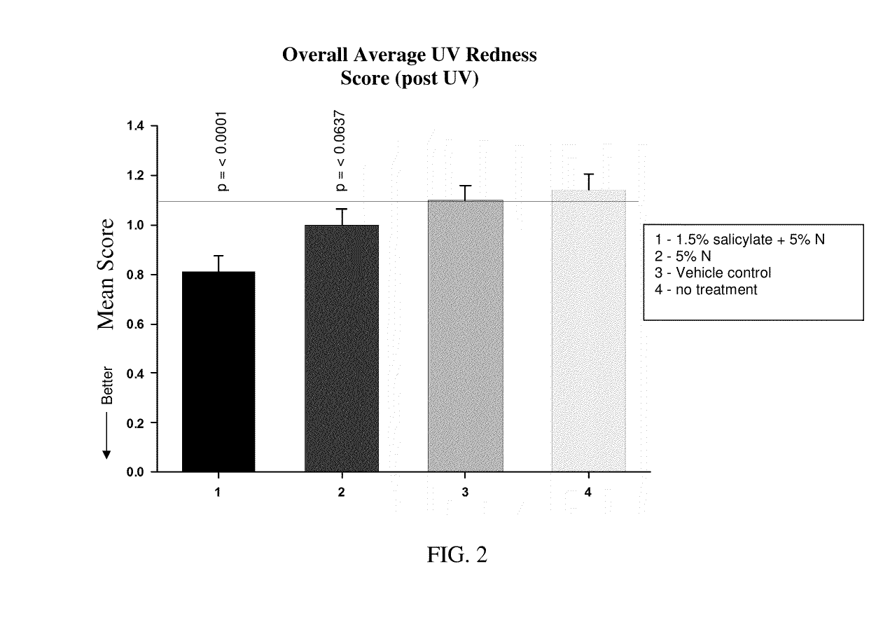

FIG. 2 illustrates the average UV redness score for days 17-30. As shown in FIG. 2, the niacinamide/salicylate containing composition provided the best overall average redness score. Thus, over the course of about two weeks, the niacinamide/salicylate containing composition provided a better erythema benefit than the other compositions after a 1.5 MED. In some instances, a UV protection benefit is demonstrated when the untreated site and/or the site treated with the vehicle only have a UV redness score and/or average UV redness score that is from 5% to 50% greater than the site treated with the vitamin B₃/salicylate containing composition (e.g., from 10% to 40%, from 15% to 30% or even about 20%).

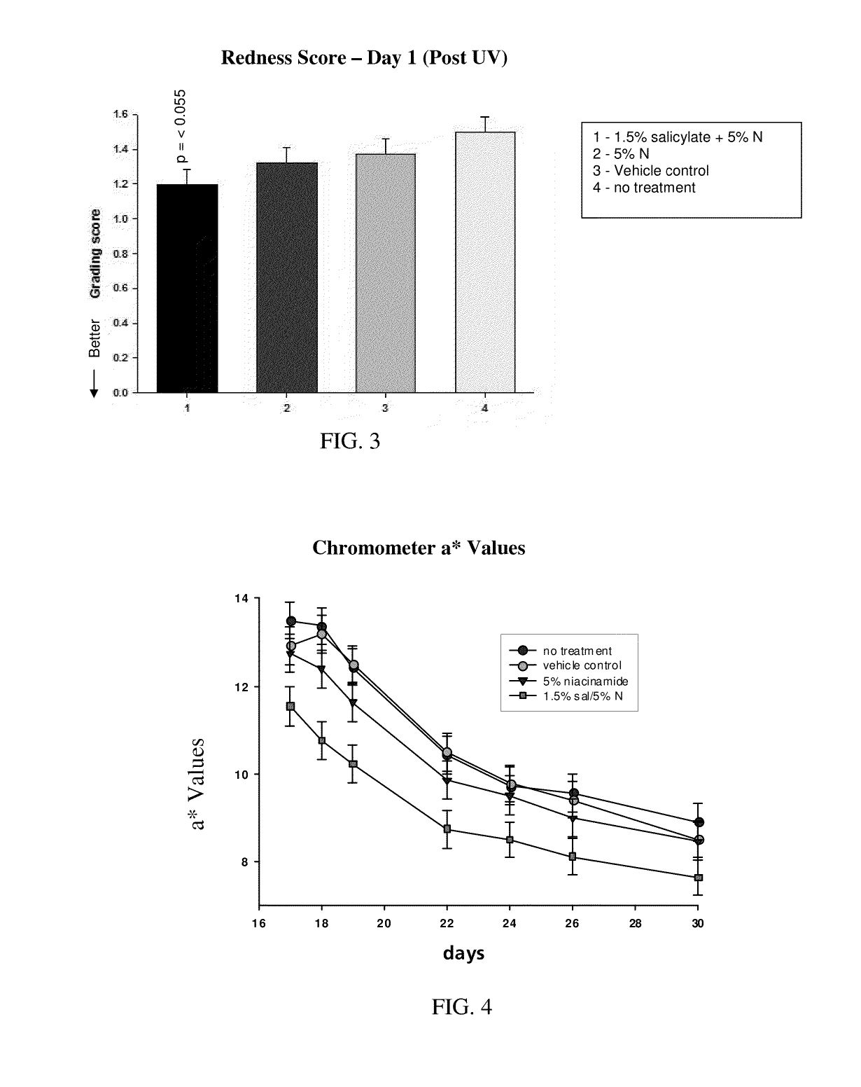

FIG. 3 illustrates the UV redness for each treatment site one day after exposure to UV (i.e., day 17). As shown in FIG. 3, the niacinamide/salicylate containing composition provided the best protection against UV induced erythema.

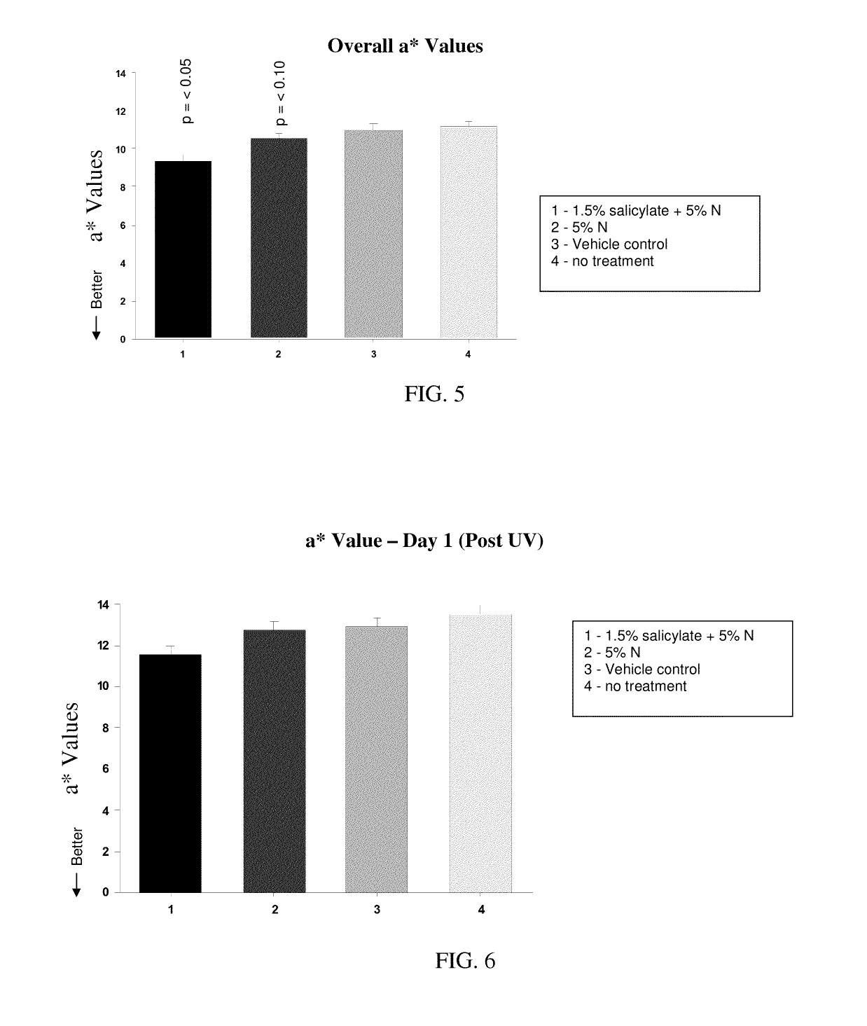

FIG. 4 illustrates the a* Value (from the CIE L*a*b* color scale) for each of the four treatment sites from days 17 to 30. The a* Values were measured with a Minolta Chroma Meter® CR300 using conventional techniques known in the art. A higher a* Value indicates that the measured image contains more red compared to an image with a lower a* Value. For purposes of this test, a lower a* Value is desired since it corresponds to less erythema. As can be seen in FIG. 4, the composition containing niacinamide and salicylate provides a lower a* Value than the other compositions tested. FIG. 5 illustrates the overall average a* Values from each of the four treatment sites derived from the data in FIG. 4. As can be seen in FIG. 5, the niacinamide/salicylate composition provides a lower average a* Value than the other compositions or the untreated site. In some instances, a UV protection benefit is demonstrated when the untreated site and/or the site treated with the vehicle only has an a* Value and/or average a* Value that is from 5% to 50% greater than the site treated with the vitamin B₃/salicylate containing composition (e.g., from 10% to 40%, from 15% to 30% or even about 20%).

FIG. 6 illustrates the a* Value for each treatment site one day after exposure to UV (i.e., day 17). As shown in FIG. 6, use of the niacinamide/salicylate containing composition prior to exposure to the UV resulted in a lower a* Value than the other compositions or no treatment, which corresponds to better protection from UV induced erythema.

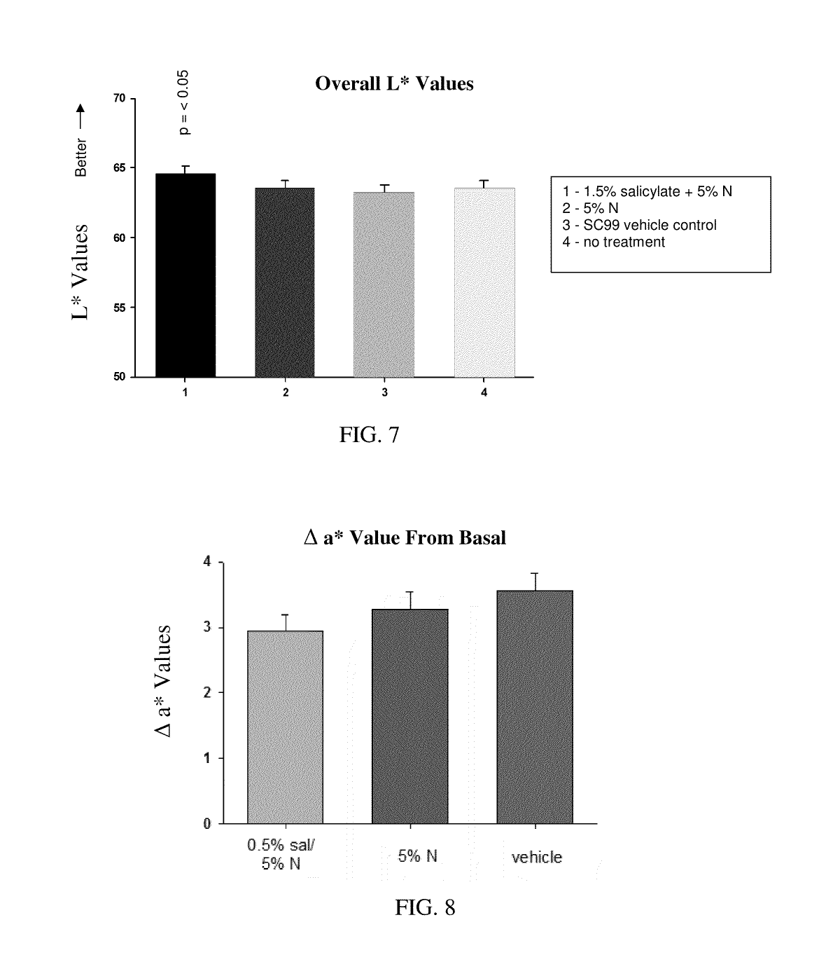

FIG. 7 illustrates the L* Value (from the CIE L*a*b* color scale) for each of the four treatment sites from days 17 to 30. The L* Values were measured with a Minolta Chroma Meter® CR300 using conventional techniques that are known in the art. L* Value indicates the lightness of an image on a scale of zero to one hundred. An L* Value of 100 indicates that the image is white, while an L* Value of 0 indicates that the image is black. In this example, a higher L* Value is associated with less UV induced erythema, and therefore more desirable. As can be seen in FIG. 7, the composition containing niacinamide and salicylate provides a better L* Value than the other compositions tested.

### Example 3

### Clinical Study II

The clinical study in this example is substantially the same as that in Example 2, except that the niacinamide/salicylate containing composition includes 0.5% salicylate instead of 1.5%. This objective of the study is to assess the ability of a composition comprising niacinamide and 0.5% salicylate to prevent and/or reduce UV-induced changes in the skin.

FIG. 8 illustrates the change in a* Value between day 1 and day 30 for each of three compositions and an untreated test site. In this example, each of three treatment sites was treated with one of the following compositions: (i) vehicle, (ii) vehicle+5% niacinamide, and (iii) vehicle+5% niacinamide+0.5% salicylate (Composition A in Table 1). The fourth treatment site was not treated. As illustrated in FIG. 8, the composition containing the niacinamide and salicylate provided the lowest change in a* Value from Basal, which corresponds to improved protection from UV induced erythema.

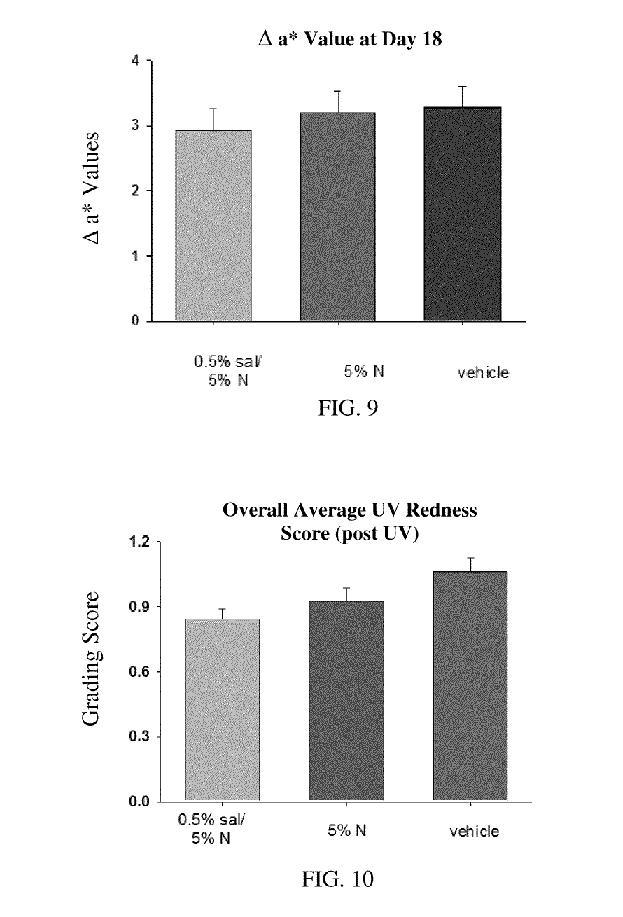

FIG. 9 illustrates the change in a* Value on day 18 (i.e., two days after a 1.5 MED UV exposure). As can be seen in FIG. 9, the salicylate/niacinamide containing composition provides better protection against UV induced erythema than the other two compositions and the no treatment site.

FIG. 10 illustrates the average UV redness score for days 17-30. As shown in FIG. 10, the niacinamide/salicylate containing composition provided the best overall average redness score.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Additionally, properties described herein may include one or more ranges of values. It is to be understood that these ranges include every value within the range, even though the individual values in the range may not be expressly disclosed.

All documents cited in the Detailed Description of the Invention are, in relevant part, incorporated herein by reference. In particular, U.S. Provisional Application Ser. No. 61/856,972 is incorporated herein by reference in its entirety. The citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of protecting skin from an environmental stressor, the method comprising:
a. identifying a target skin portion where protection from environmental stress is desired;
b. topically applying a skin care composition to the target skin portion, wherein the skin care composition includes
i. a safe and effective amount of a vitamin B₃ compound,
ii. a safe and effective amount of a salicylate;
iii. an organic sunscreen active,
iv. a dermatologically acceptable carrier, and
v. a pH of between about 5 and about 7; and
c. leaving the skin care composition on the target skin portion for a sufficient amount of time for the vitamin B₃ compound and salicylate to provide protection from the environmental stressor.

2. The method of claim 1, wherein the salicylate is sodium salicylate.

3. The method of claim 1, wherein the vitamin B₃ compound is niacinamide.

4. The method of claim 1, wherein the pH is from about 5.0 to about 6.0.

5. The method of claim 1, wherein the composition is applied to the target skin portion at least once per day at least four days per week for at least two weeks.

6. The method of claim 1, wherein the environmental stressor selected from the group consisting of ultraviolet radiation (UV), cigarette and cigar smoke, ozone, combustible engine exhaust, smog, surfactants, and radiation from a computer, telephone, or television monitor.

7. The method of claim 1, wherein the target skin portion exhibits less environmental stressor induced damage than an untreated skin portion when both portions of skin are exposed to the same type and amount of the environmental stressor and between about 12 and about 24 hours have passed since the skin care composition was applied to the target skin portion.

8. The method of claim 7, wherein the environmental stressor induced damage is quantified and the untreated skin portion exhibits at least 10% more damage than the treated skin portion.

9. The method of claim 1, wherein the skin care composition has a Sun Protection Factor of between about 5 and 50.

10. The method of claim 1, wherein the skin care composition includes an additional skin care active.

11. A method of protecting skin from UV induced erythema, the method comprising:
a. identifying a target skin portion where protection from UV induced erythema is desired;
b. topically applying a skin care composition to the target skin portion, wherein the skin care composition includes
i. a safe and effective amount of niacinamide,
ii. a safe and effective amount of a salicylate,
iii. an organic sunscreen active,
iv. a dermatologically acceptable carrier, and
v. a pH of between about 5 and about 7; and
c. allowing the skin care composition to remain on the target skin portion for a sufficient amount of time for the niacinamide and salicylate to provide protection from erythema.

12. The method of claim 11, wherein the target skin portion exhibits less erythema than an untreated skin portion when both portions of skin are exposed to a 1.5 MED of UV and between about 12 and about 24 hours have passed since the composition was applied to the target skin area.

13. The method of claim 12, wherein the erythema exhibited is quantified by measuring an a* Value of the treated and untreated portions of skin and the a* Value of the untreated skin portion is at least 10% greater than the a* Value of treated skin portion.

14. The method of claim 12, wherein the erythema exhibited is quantified by measuring a Redness Score of the treated and untreated portions of skin and the Redness Score of the untreated skin portion is at least 10% greater than the treated skin portion.

15. The method of claim 11, wherein the target skin portion exhibits less environmental stressor induced damage than an untreated skin portion when both portions of skin are exposed to the same type and amount of the environmental stressor.

16. The method of claim 11, wherein the pH is from about 5.0 to about 6.0.

17. A method of protecting skin from UV induced erythema, the method comprising:
a. identifying a target skin portion where protection from UV induced erythema is desired;
b. topically applying a skin care composition to the target skin portion, wherein the skin care composition includes
i. a safe and effective amount of niacinamide,
ii. a safe and effective amount of a salicylate derivative selected from the group consisting of 3,4-dihydroxybenzoate, 2,5-dihydroxybenzoate, 2,6-dihydroxybenzoate, and salts of rhein,
iii. an organic sunscreen active,
iv. a dermatologically acceptable carrier, and
v. a pH of between about 5 and about 7; and
c. allowing the skin care composition to remain on the target skin portion for a sufficient amount of time for the niacinamide and salicylate to provide protection from erythema.
